(19) United States
(12) Patent Application Publication
(10) Pub. No.: US 2024/0301098 A1
(43) Pub. Date: Sep. 12, 2024

(54) THE OXIDATION OF CARBON-HYDROGEN BONDS OF POLYMERS USING OZONE

(71) Applicant: P2 Science, Inc., Woodbridge, CT (US)

(72) Inventor: Patrick FOLEY, New Haven, CT (US)

(21) Appl. No.: 18/550,087

(22) PCT Filed: Mar. 10, 2022
(86) PCT No.: PCT/US2022/019811

§ 371 Date: Sep. 11, 2023

(51) Int. Cl. C08F 8/06; C07C 45/27; C07C 49/784; C07C 51/34; C07C 57/38; C07C 59/84; C08F 112/08

(52) U.S. Cl. CPC C08F 8/06; C07C 45/27; C07C 49/784; C07C 51/34; C07C 57/38; C07C 59/84; C08F 112/08

(57) ABSTRACT

The present disclosure is directed to novel methods of oxidizing polymers, such as polystyrene, using ozone, and deconstructing such polymers using ozone, to provide oligomeric product compounds, compounds made according to said methods, homopolymers and heteropolymers derived from said compounds, and compositions comprising said homopolymers and heteropolymers.

### FIELD OF INVENTION

The present disclosure is directed to novel methods of oxidizing polymers, such as polystyrene, using ozone, and deconstructing such polymers using ozone, to provide oligomeric product compounds, compounds made according to said methods, homopolymers and heteropolymers derived from said compounds, and compositions comprising said homopolymers and heteropolymers.

### BACKGROUND

The controlled oxidation of hydrocarbons is a difficult chemical process to regulate. The oxidation of C—H bonds in hydrocarbons is particularly challenging because the energetic barriers to such reactions are relatively large, requiring catalysts, expensive reagents, and/or high temperatures. Conditions must be tailored so enough energy is available for an appreciable rate of chemical oxidation while minimizing inevitable but undesirable side reactions.

There are a wide variety of polymers used in society today, however, the vast majority of them, especially hydrocarbon polymers, are not biodegradable and are highly persistent in the environment. Efforts at encouraging consumer and business recycling have had varied results in different countries and for different polymers.

One such polymer of particular interest is polystyrene (PS), a petrochemical-derived polymer that is widely used and constitutes ˜8% of all global polymer production. Its beneficial properties include low specific weight, good optical properties, mechanical flexibility, and high chemical resistance. Despite these desirable properties it is recycled at a rate of only 1% and is frequently lost to the environment where it is resistant to degradation, biological or otherwise. It would therefore be highly desirable to identify new chemical pathways that could deconstruct PS post-use and, ideally, convert it into a more useful polymer intermediate with improved properties.

Other polymers of particular challenge include polyethylene, polypropylene, polyamides (e.g., nylon), polyvinyl chloride (PVC), and polyvinylidene chloride (PVDC).

The challenge and the opportunity in this regard lie in the polymeric structure. Like most synthetic hydrocarbon polymers, the polystyrene backbone is formed through radical polymerization of monomeric alkenes (e.g., styrene) to generate highly stable, repeating C—C bonds. While these bonds are very stable and uniform, they also make the polymer highly recalcitrant and resistant to deconstruction by most biological, mechanical, and even chemical methods.

Science has already explored various thermal and autoxidative methods for decomposing polystyrene, primarily as a means of destroying used polystyrene products. Unlike these approaches, however, the inventors are interested in developing polymer degradation methods that provide well defined product profiles with functional groups that can be used to enhance function, recyclability, and biodegradability, all while using only electricity and air as the ultimate primary reagents.

Ozone (O₃) is a powerful and selective oxidant, but has yet seen little use in the field of oxidation of saturated hydrocarbons. Ozone is most commonly associated with the oxidation of carbon-carbon double and triple bonds, which proceed through the unique Crigee addition/rearrangement mechanism yielding cyclic oxygenated intermediates, and ultimately cleavage of the substrate. Such mechanisms would not be operable for the oxidation of the C—H bond of a saturate hydrocarbon.

A few examples have been reported of the direct oxidation of alkanes to alcohols and/or ketones using ozone, but the yields are generally either undetermined (for lack of isolation and purification of a product) or low absent the addition of catalysts. For example, it has been reported that yields of such oxidations may improve in the presence of acidic catalysts, silica gel and/or activated charcoal catalysts, metal catalysts (e.g., copper, manganese and iron), or UV irradiation.

In addition, typical hydrocarbon oxidation conditions generate result in peroxide intermediates, which must then be reduced to yield the desired hydroxy and carbonyl products. Furthermore, existing reaction conditions often require high temperatures, have long reaction times, have poor yields, or require expensive reagents (e.g., stoichiometric metal oxidants). It is also difficult to control these reactions, leading to overoxidation, distributions of structural isomers, and undesired chemical side-reactions and by-products (which can be difficult to remove by traditional purification means).

Some research has already demonstrated the ozone-mediated surface oxidation of solid polystyrene, but not oxidative deconstruction of polystyrene polymer. Kefeli et al. (“Ratios of rates of the reaction of ozone with polystyrene on the surface and in the volume of the polymer,” Polymer Science U.S.S.R 1976, 18(3), 695-701) first looked at the surface kinetics of the ozone-mediated oxidation of polystyrene. They posited that in the absence of UV irradiation, more than 98% of the reaction took place at the sp3-hybridized tertiary methylene on the back bone of the polystyrene polymer, but they provided little information as to what functional groups resulted or how the reaction proceeded. This surface chemistry was further explored by additional groups who sought to modify the polystyrene surfaces with ozone in the presence of UV irradiation to derive more hydrophilic polystyrene derivatives. The mechanisms elucidated in these latter studies suggested that the ozone instead reacted with the aromatic rings on the polystyrene periphery due to the UV irradiation, as well as on the backbone, and that both reactions generated carboxylic acids and carbonyls. The invention herein reveals a powerful and unexpected way to harness these phenomena to deconstruct and upcycle the PS polymer.

It would be extremely beneficial to society for there to be a commercially viable method for the direct oxidation of synthetic polymers, especially hydrocarbon polymers, such as polystyrene. An efficient, fast, selective, and simple (low-cost) process is currently needed for the oxidation of such polymers that doesn't rely on free-radical oxidation with O₂.

The inventors have applied their expertise in ozonolysis chemistry and reactor technology to further develop the state of knowledge of the prior art in order to provide such commercially viable methods for oxidative deconstruction and upcycling of polymers such as polystyrene.

### BRIEF SUMMARY OF THE INVENTION

It has been unexpectedly found that it is possible to selectively activate the methylene C—H bond adjacent to the aromatic ring on the back bone of the polystyrene polymer using ozone, in such a way that it will result in a scission event that yields carboxylic acid products and carbonyl products, in high, reproducible and controllable yield, thereby achieving simultaneous deconstruction and functionalization. This unexpected and beneficial invention reveals a powerful and selective pathway to upcycle polystyrene using a clean chemical reagent, ozone, which is derived from electricity.

The present disclosure provides a method for the selective oxidation of the tertiary hydrogen atoms of the polystyrene backbone using ozone resulting in the deconstructions of the polymer. Without being bound by theory, it is believed that these methods would also be effective for oxidizing and deconstructing other synthetic polymers, particularly hydrocarbon polymers.

In a first aspect, the present disclosure provides a method of oxidizing the tertiary carbon of a polymer using ozone, comprising the steps of (1) exposing the polymer to ozone, optionally in an aqueous and/or non-aqueous solvent, and (2) isolating or purifying the resulting product or products. In some embodiments, the products are a mixture of carbonyl compounds (e.g., ketones) and carboxylic acid compounds.

In a second aspect, the present disclosure provides a method of preparing at least one carbonyl compound (e.g., a ketone) or carboxylic acid from a polymer, comprising the steps of (1) exposing the polymer to ozone, optionally in an aqueous and/or non-aqueous solvent, and (2) isolating or purifying the resulting carbonyl and/or carboxylic acid product or products.

In a third aspect, the present disclosure provides a method of oxidatively decomposing polymers to carbonyl and carboxylic acid degradation products, comprising the steps of (1) exposing the polymer to ozone, optionally in an aqueous and/or non-aqueous solvent, and optionally (2) isolating or purifying the resulting product or products.

In another aspect, the present disclosure compounds produced according to the methods disclosed herein.

### DETAILED DESCRIPTION OF THE INVENTION

The inventors have developed advanced tools for process intensified gas liquid reactions with specific expertise in ozone chemistry. See, e.g., U.S. Pat. Nos. 10,071,944, and 10,668,446, the contents of each of which are hereby incorporated by reference in their entireties. Such process intensification allows for the highly controlled and stoichiometric dosing of ozone to a liquid film which enables selectivity and precise temperature control. With such high degrees of control, process parameterization polystyrene upcycling can be achieved.

Using polystyrene as an example, the oxidation process may be summarized as shown in the following scheme:

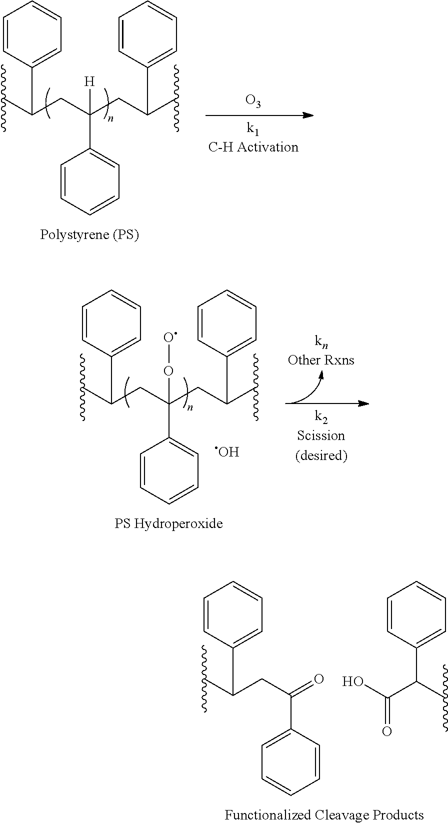

Without being bound by theory, it believed that the reaction proceeds through a hydroperoxide intermediate (e.g., a hydroperoxide radical), which may decompose to the desired carbonyl and carboxylic acid products through k₂, or which may participate in off-path oxidation pathways denoted by kn.

It is understood that this oxidative cleavage takes places at numerous locations along the polystyrene backbone, resulting in a variety of bifunctional linear oligomeric products, for example:

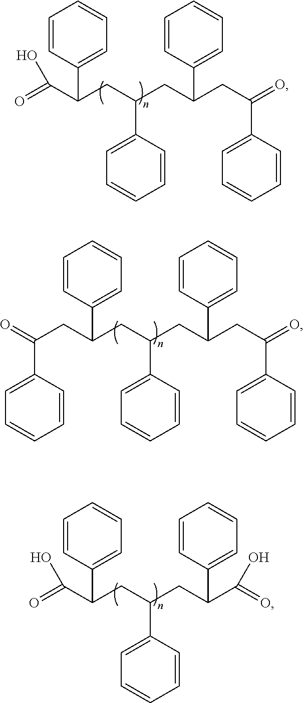

wherein n can have any integer value from 0 up to 100,000, or more. More typically, the reaction products will have n values varying from 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof, depending on the reaction conditions (e.g., temperature, ozone concentration, reaction time, polystyrene concentration).

The products formed from the oxidative deconstruction of the polymers, e.g., of polystyrene, can potentially be used ‘as-is’ with benefits including higher hydrophilicity and biodegradability. Further, the deconstructed polymer products (oligomers) contain functional groups (ketone and/or carboxylic acid) that can make them useful as intermediates for making new polymers. In some embodiments, the products include oligomeric dicarboxylic acids. A high content of dicarboxylic acids in the product mix would allow for repolymerization to make polyesters and polyamides, but reacting the dicarboxylic acids with suitable diamines or dialcohols. These new polymer-derived polyesters and polyamides could further incorporate renewable feedstocks such as bio-based PDO, glycerol, and various carbohydrates among others. These novel polymers will have enhanced biodegradability, renewability, and recyclability; all key features of successful polymer upcycling.

Without wishing to be bound by theory, the diacid, diketone and acid-ketone oligomers resulting from the methods may be very useful as dispersants, foam containers, packing peanuts, structural packing, biological containers for cell and tissue growth, food wrappers, 3-D printing substrates, as a vehicle or additive for coatings, paints, lubricants, molded plastics, synthetic fibers, and personal care products such as hair products and cosmetics.

Further the oligomeric product monoacids, diacids, and carbonyl compounds may be further functionalized and/or repolymerized with amines and/or alcohols to make additional functional materials. Examples include using diamines and/or other polyamines to make polyamides and/or polyimines; using diols and/or other polyols to make polyesters and/or alcohols; using amino-alcohols to make mixed esters, amides, and imines; and using any of the above combinations to make epoxy and/or polyurethane resins. To the extent these alcohols and imines can be renewably derived they can likely further add value and biocompatibility to the end products.

In a first aspect, the present disclosure provides a method (Method 1) of oxidizing the tertiary carbon (e.g., one or more tertiary carbons) of a polymer (e.g., a hydrocarbon polymer, such as polystyrene) using ozone, comprising the steps of (1) exposing the polymer to ozone, optionally in an aqueous and/or non-aqueous solvent, and (2) isolating or purifying the resulting product or products. In some embodiments, the products are a mixture of carbonyl compounds (e.g., ketones) and carboxylic acid compounds. In some embodiments, ozone is the only oxidant added to the reaction (i.e., ozone is the sole oxidizing agent).

In further embodiments of the first aspect, the present disclosure provides as follows:
1.1 Method 1, wherein the polymer is a synthetic polymer, e.g., a hydrocarbon polymer, optionally a saturated hydrocarbon, a chlorinated hydrocarbon, or a polyamide (i.e., not a peptide polymer).
1.2 Method 1.1, wherein the polymer is a polyethylene, polypropylene, polystyrene, aliphatic polyamide (e.g., nylon, such as nylon-6,6), or a polyvinyl chloride or polyvinylidene chloride.
1.3 Method 1.2, wherein the polymer is polystyrene.
1.4 Method 1 or any of 1.1-1.3, wherein the step (1) of exposing the polymer (e.g., polystyrene) to ozone comprises exposing the polymer to an ozone/oxygen mixture or ozone/nitrogen mixture or ozone/air mixture.
1.5 Method 1 or any of 1.1-1.4, wherein step (1) is carried out in the absence of any other added oxidants or oxidizing agents.
1.6 Any preceding method, wherein step (1) does not comprise the presence or addition of any catalyst (e.g., any metal, activated charcoal, or silica gel).
1.7 Any preceding method, wherein step (1) occurs in the dark (e.g., the reaction occurs without exposure to light, e.g., UV light).
1.8 Any preceding method, wherein in step (1) the polymer (e.g., polystyrene) is dissolved or suspended in an aqueous solution or emulsion, optionally in an acidic (i.e., pH<7) or alkaline (e.g., pH>7) aqueous solution or emulsion.
1.9 Method 1.8, wherein the polymer (e.g., polystyrene) is dissolved or suspended in an alkaline aqueous solution, optionally wherein the alkaline agent is an inorganic base (e.g., an alkoxide, hydroxide, oxide, carbonate or bicarbonate of an alkali or alkaline earth metal).
1.10 Method 1.9, wherein the polymer (e.g., polystyrene) is dissolved or suspended in an aqueous solution of a sodium, potassium, lithium, calcium or magnesium hydroxide, alkoxide, oxide, carbonate or bicarbonate (e.g., sodium hydroxide or potassium hydroxide).
1.11 Method 1.9 or 1.10, wherein the aqueous solution or emulsion has a pH from 7.5 to 12, or from 8 to 12, or from 9 to 11, or from 9 to 10.
1.12 Any preceding method, wherein the polymer (e.g., polystyrene) is dissolved or suspended in a mixture of an aqueous solution and an organic co-solvent (such as an alcohol, ester, or ether solvent, e.g., methanol, ethanol, propanol, THF, or MTBE).
1.13 Any preceding method, wherein the products of the reaction (e.g., the polystyrene oligomers or carbonyl or carboxylic acid products) are obtained directly from the reaction between the polymer (e.g., polystyrene) and the ozone (e.g., no intermediate partially oxidized or oxidized species are formed or isolated).
1.14 Any preceding method, wherein the method does not comprise the formation of any alkyl peroxide intermediate.
1.15 Any preceding method, wherein the method does not comprise any step comprising a reducing agent between step (1) and step (2).
1.16 Method 1 or any of 1.1-1.15, wherein the method is a batch method.
1.17 Method 1 or any of 1.1-1.15, wherein the method is a continuous flow method, e.g., wherein the method is performed in a flow reactor.
1.18 Method 1 or any of 1.1-1.15, wherein the method is performed in one or more of a falling film reactor, a batch reactor, a continuous stirred-tank reactor, and/or loop reactor, either individually or in series.
1.19 Method 1.18, wherein the method is performed in one or more falling film reactors, e.g., multi-tube falling film reactors, optionally in series and optionally with recirculation, for example, as described in any embodiment of U.S. Pat. No. 10,071,944.
1.20 Any preceding method, wherein step (2) comprises separating the product or products from the reaction solvent, or from the ozone, or both.
1.21 Any preceding method, wherein step (2) comprises distillation, fractional distillation, chromatography, crystallization or a combination thereof.
1.22 Any preceding method, wherein the polymer is polystyrene, and the method does not result in oxidation of either the methylene carbon atoms of the polystyrene backbone or any of the phenyl ring carbons of the polystyrene polymer.
1.23 Any preceding method, wherein the polymer is polystyrene, and wherein the product or products of the reaction comprises a compound of the formula:

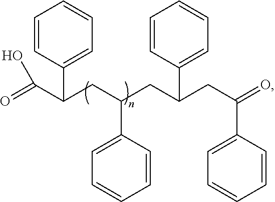

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
1.24 Any preceding method, wherein the polymer is polystyrene, and wherein 1.24 the product or products of the reaction comprises a compound of the formula:

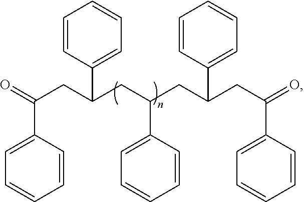

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof
1.25 Any preceding method, wherein the polymer is polystyrene, and wherein 1.25 the product or products of the reaction comprises a compound of the formula:

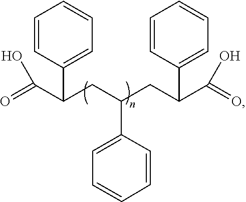

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
1.26 Method 1.23, wherein the method further comprises the step (3) of reducing the polystyrene oligomer's ketone groups to secondary alcohol groups.
1.27 Method 1.26, wherein the method further comprises the step (4) of reacting the resulting polystyrene oligomer acid-alcohols with a reagent to promote condensation and polymerization to form a homopolymeric polyester, and optionally isolating and/or purifying said polyester.
1.28 Method 1.26, wherein the method further comprises the step (4) of reacting the resulting polystyrene oligomer acid-alcohols with carboxylic acid/alcohol monomer (e.g., a terminal hydroxy alkane carboxylic acid) and a reagent to promote condensation and polymerization to form a heteropolymeric polyester, and optionally isolating and/or purifying said polyester.
1.29 Method 1.26, wherein the method further comprises the step (4) of reacting the resulting polystyrene oligomer acid-alcohols with carboxylic acid/amine monomer (e.g., a terminal amino alkane carboxylic acid) and a reagent to promote condensation and polymerization to form a heteropolymeric polyester-polyamide mixed copolymer, and optionally isolating and/or purifying said copolymer.
1.30 Method 1.24, wherein the method further comprises the step (3) of reducing the polystyrene oligomer's ketone groups to secondary alcohol groups.
1.31 Method 1.30, wherein the method further comprises the step (4) of reacting the resulting polystyrene oligomer dialcohol with dicarboxylic acid monomers (e.g., terminal carboxy alkane carboxylic acid) and a reagent to promote condensation and polymerization to form a heteropolymeric polyester, and optionally isolating and/or purifying said polyester.
1.32 Method 1.25, wherein the method further comprises the step (3) of polymerization to form a heteropolymeric polyester, and optionally isolating and/or purifying said polyester;
1.33 Method 1.25, wherein the method further comprises the step (3) of reacting the resulting polystyrene oligomer diacid with a diamine monomer (e.g., a bis-terminal amino alkane) and a reagent to promote condensation and polymerization to form a heteropolymeric polyamide, and optionally isolating and/or purifying said polyamide.
1.34 Method 1.25, wherein the method further comprises the step (3) of reacting the resulting polystyrene oligomer diacid with an amino-alcohol monomer (e.g., a terminal amino alkane alcohol) and a reagent to promote condensation and polymerization to form a heteropolymeric mixed polyamide-polyester copolymer, and optionally isolating and/or purifying said copolymer.
1.35 Method 1.23, 1.24 or 1.25, further comprising any one or more steps necessary to form heteropolymeric polyimines or mixed polyester-polyimines, polyamide-polyimines, polyurethanes, or other heteropolymers.
1.36 Any preceding method, wherein the method further comprises one or more steps immediately following step (1) and before step (2), or after step (2) and before any subsequent steps, selected from peroxide passivation, further oxidation, downstream derivatization, catalytic scission, and catalytic rearrangement.
1.37 Any preceding method, wherein the ozone is generated using an ozone generator from an oxygen feed, and optionally wherein said oxygen is derived from the hydrolysis of water.

In a second aspect, the present disclosure provides a method (Method 2) of preparing at least one carbonyl compound (e.g., a ketone) or carboxylic acid from a polymer (e.g., a hydrocarbon polymer, such as polystyrene), comprising the steps of (1) exposing the polymer to ozone, optionally in an aqueous and/or non-aqueous solvent, and (2) isolating or purifying the resulting carbonyl and/or carboxylic acid product or products.

In further embodiments of the second aspect, the present disclosure provides as follows:
2.1 Method 2, wherein the polymer is a synthetic polymer, e.g., a hydrocarbon polymer, optionally a saturated hydrocarbon, a chlorinated hydrocarbon, or a polyamide (i.e., not a peptide polymer).
2.2 Method 2.1, wherein the polymer is a polyethylene, polypropylene, polystyrene, aliphatic polyamide (e.g., nylon, such as nylon-6,6), or a polyvinyl chloride or polyvinylidene chloride.
2.3 Method 2.2, wherein the polymer is polystyrene.
2.4 Method 2 or any of 2.1-2.3, wherein the step (1) of exposing the polymer (e.g., polystyrene) to ozone comprises exposing the polymer to an ozone/oxygen mixture or ozone/nitrogen mixture or ozone/air mixture.
2.5 Method 2, or any of 2.1-2.4, wherein step (1) is carried out in the absence of any other added oxidants or oxidizing agents.
2.6 Any preceding method, wherein step (1) does not comprise the presence or addition of any catalyst (e.g., any metal, activated charcoal, or silica gel).
2.7 Any preceding method, wherein step (1) occurs in the dark (e.g., the reaction occurs without exposure to light, e.g., UV light).
2.8 Any preceding method, wherein in step (1) the polymer (e.g., polystyrene) is dissolved or suspended in an aqueous solution or emulsion, optionally in an acidic (i.e., pH<7) or alkaline (e.g., pH>7) aqueous solution or emulsion.
2.9 Method 2.8, wherein the polymer (e.g., polystyrene) is dissolved or suspended in an alkaline aqueous solution, optionally wherein the alkaline agent is an inorganic base (e.g., an alkoxide, hydroxide, oxide, carbonate or bicarbonate of an alkali or alkaline earth metal).
2.10 Method 2.9, wherein the polymer (e.g., polystyrene) is dissolved or suspended in an aqueous solution of a sodium, potassium, lithium, calcium or magnesium hydroxide, alkoxide, oxide, carbonate or bicarbonate (e.g., sodium hydroxide or potassium hydroxide).
2.11 Method 2.9 or 2.10, wherein the aqueous solution or emulsion has a pH from 7.5 to 12, or from 8 to 12, or from 9 to 11, or from 9 to 10.
2.12 Any preceding method, wherein the polymer (e.g., polystyrene) is dissolved or suspended in a mixture of an aqueous solution and an organic co-solvent (such as an alcohol, ester, or ether solvent, e.g., methanol, ethanol, propanol, THF, or MTBE).
2.13 Any preceding method, wherein the products of the reaction (e.g., the polystyrene oligomers or carbonyl or carboxylic acid products) are obtained directly from the reaction between the polymer (e.g., polystyrene) and the ozone (e.g., no intermediate partially oxidized or oxidized species are formed or isolated).
2.14 Any preceding method, wherein the method does not comprise the formation of any alkyl peroxide intermediate.
2.15 Any preceding method, wherein the method does not comprise any step comprising a reducing agent between step (1) and step (2).
2.16 Method 2 or any of 2.1-2.15, wherein the method is a batch method.
2.17 Method 2 or any of 2.1-2.15, wherein the method is a continuous flow method, e.g., wherein the method is performed in a flow reactor.
2.18 Method 2 or any of 2.1-2.15, wherein the method is performed in one or more of a falling film reactor, a batch reactor, a continuous stirred-tank reactor, and/or loop reactor, either individually or in series.
2.19 Method 2.18, wherein the method is performed in one or more falling film reactors, e.g., multi-tube falling film reactors, optionally in series and optionally with recirculation, for example, as described in any embodiment of U.S. Pat. No. 10,071,944.
2.20 Any preceding method, wherein step (2) comprises separating the product or products from the reaction solvent, or from the ozone, or both.
2.21 Any preceding method, wherein step (2) comprises distillation, fractional distillation, chromatography, crystallization or a combination thereof.
2.22 Any preceding method, wherein the polymer is polystyrene, and wherein the method does not result in oxidation of either the methylene carbon atoms of the polystyrene backbone or any of the phenyl ring carbons of the polystyrene polymer.
2.23 Any preceding method, wherein the polymer is polystyrene, and wherein the product or products of the reaction comprises a compound of the formula:

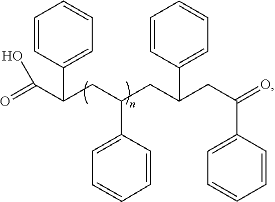

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
2.24 Any preceding method, wherein the polymer is polystyrene, and wherein 2.24 the product or products of the reaction comprises a compound of the formula:

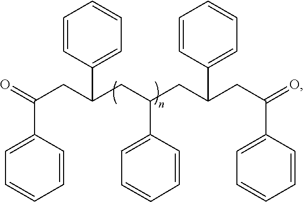

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
2.25 Any preceding method, wherein the polymer is polystyrene, and wherein 2.25 the product or products of the reaction comprises a compound of the formula:

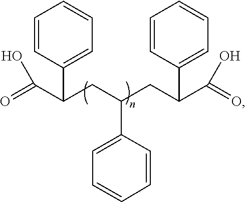

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
2.26 Method 2.23, wherein the method further comprises the step (3) of reducing the polystyrene oligomer's ketone groups to secondary alcohol groups.
2.27 Method 2.26, wherein the method further comprises the step (4) of reacting the resulting polystyrene oligomer acid-alcohols with a reagent to promote condensation and polymerization to form a homopolymeric polyester, and optionally isolating and/or purifying said polyester.
2.28 Method 2.26, wherein the method further comprises the step (4) of reacting the resulting polystyrene oligomer acid-alcohols with carboxylic acid/alcohol monomer (e.g., a terminal hydroxy alkane carboxylic acid) and a reagent to promote condensation and polymerization to form a heteropolymeric polyester, and optionally isolating and/or purifying said polyester.
2.29 Method 2.26, wherein the method further comprises the step (4) of reacting the resulting polystyrene oligomer acid-alcohols with carboxylic acid/amine monomer (e.g., a terminal amino alkane carboxylic acid) and a reagent to promote condensation and polymerization to form a heteropolymeric polyester-polyamide mixed copolymer, and optionally isolating and/or purifying said copolymer.
2.30 Method 2.24, wherein the method further comprises the step (3) of reducing the polystyrene oligomer's ketone groups to secondary alcohol groups.
2.31 Method 2.30, wherein the method further comprises the step (4) of reacting the resulting polystyrene oligomer dialcohol with dicarboxylic acid monomers (e.g., terminal carboxy alkane carboxylic acid) and a reagent to promote condensation and polymerization to form a heteropolymeric polyester, and optionally isolating and/or purifying said polyester.
2.32 Method 2.25, wherein the method further comprises the step (3) of reacting the resulting polystyrene oligomer diacid with a dialcohol monomer (e.g., a bis-terminal hydroxy alkane) and a regent to promote condensation and polymerization to form a heteropolymeric polyester, and optionally isolating and/or purifying said polyester;
2.33 Method 2.25, wherein the method further comprises the step (3) of reacting the resulting polystyrene oligomer diacid with a diamine monomer (e.g., a bis-terminal amino alkane) and a reagent to promote condensation and polymerization to form a heteropolymeric polyamide, and optionally isolating and/or purifying said polyamide.
2.34 Method 2.25, wherein the method further comprises the step (3) of reacting the resulting polystyrene oligomer diacid with an amino-alcohol monomer (e.g., a terminal amino alkane alcohol) and a reagent to promote condensation and polymerization to form a heteropolymeric mixed polyamide-polyester copolymer, and optionally isolating and/or purifying said copolymer.
2.35 Method 2.23, 2.24 or 2.25, further comprising any one or more steps necessary to form heteropolymeric polyimines or mixed polyester-polyimines, polyamide-polyimines, polyurethanes, or other heteropolymers.
2.36 Any preceding method, wherein the method further comprises one or more steps immediately following step (1) and before step (2), or after step (2) and before any subsequent steps, selected from peroxide passivation, further oxidation, downstream derivatization, catalytic scission, and catalytic rearrangement.
2.37 Any preceding method, wherein the ozone is generated using an ozone generator from an oxygen feed, and optionally wherein said oxygen is derived from the hydrolysis of water.

In a third aspect, the present disclosure provides a method (Method 3) of oxidatively decomposing a polymer (e.g., a hydrocarbon polymer, such as polystyrene) to carbonyl and carboxylic acid degradation products, comprising the steps of (1) exposing the polymer to ozone, optionally in an aqueous and/or non-aqueous solvent, and optionally (2) isolating or purifying the resulting product or products.

In further embodiments of the third aspect, the present disclosure provides as follows:
3.1 Method 3, wherein the polymer is a synthetic polymer, e.g., a hydrocarbon polymer, optionally a saturated hydrocarbon, a chlorinated hydrocarbon, or a polyamide (i.e., not a peptide polymer).
3.2 Method 3.1, wherein the polymer is a polyethylene, polypropylene, polystyrene, aliphatic polyamide (e.g., nylon, such as nylon-6,6), or a polyvinyl chloride or polyvinylidene chloride.
3.3 Method 3.2, wherein the polymer is polystyrene.
3.4 Method 3 or any of 3.1-3.3, wherein the step (1) of exposing the polymer (e.g., polystyrene) to ozone comprises exposing the polymer to an ozone/oxygen mixture or ozone/nitrogen mixture or ozone/air mixture.
3.5 Method 3, or any of 3.1-3.4, wherein step (1) is carried out in the absence of any other added oxidants or oxidizing agents.
3.6 Any preceding method, wherein step (1) does not comprise the presence or addition of any catalyst (e.g., any metal, activated charcoal, or silica gel).
3.7 Any preceding method, wherein step (1) occurs in the dark (e.g., the reaction occurs without exposure to light, e.g., UV light).
3.8 Any preceding method, wherein in step (1) the polymer (e.g., polystyrene) is dissolved or suspended in an aqueous solution or emulsion, optionally in an acidic (i.e., pH<7) or alkaline (e.g., pH>7) aqueous solution or emulsion.
3.9 Method 3.8, wherein the polymer (e.g., polystyrene) is dissolved or suspended in an alkaline aqueous solution, optionally wherein the alkaline agent is an inorganic base (e.g., an alkoxide, hydroxide, oxide, carbonate or bicarbonate of an alkali or alkaline earth metal).
3.10 Method 3.9, wherein the polymer (e.g., polystyrene) is dissolved or suspended in an aqueous solution of a sodium, potassium, lithium, calcium or magnesium hydroxide, alkoxide, oxide, carbonate or bicarbonate (e.g., sodium hydroxide or potassium hydroxide).
3.11 Method 3.9 or 3.10, wherein the aqueous solution or emulsion has a pH from 7.5 to 12, or from 8 to 12, or from 9 to 11, or from 9 to 10.
3.12 Any preceding method, wherein the polymer (e.g., polystyrene) is dissolved or suspended in a mixture of an aqueous solution and an organic co-solvent (such as an alcohol, ester, or ether solvent, e.g., methanol, ethanol, propanol, THF, or MTBE).
3.13 Any preceding method, wherein the products of the reaction (e.g., the polystyrene oligomers or carbonyl or carboxylic acid products) are obtained directly from the reaction between the polymer (e.g., polystyrene) and the ozone (e.g., no intermediate partially oxidized or oxidized species are formed or isolated).
3.14 Any preceding method, wherein the method does not comprise the formation of any alkyl peroxide intermediate.
3.15 Any preceding method, wherein the method does not comprise any step comprising a reducing agent between step (1) and step (2).
3.16 Method 3 or any of 3.1-3.15, wherein the method is a batch method.
3.17 Method 3 or any of 3.1-3.15, wherein the method is a continuous flow method, e.g., wherein the method is performed in a flow reactor.
3.18 Method 3 or any of 3.1-3.15, wherein the method is performed in one or more of a falling film reactor, a batch reactor, a continuous stirred-tank reactor, and/or loop reactor, either individually or in series.
3.19 Method 3.18, wherein the method is performed in one or more falling film reactors, e.g., multi-tube falling film reactors, optionally in series and optionally with recirculation, for example, as described in any embodiment of U.S. Pat. No. 10,071,944.
3.20 Any preceding method, wherein step (2) comprises separating the product or products from the reaction solvent, or from the ozone, or both.
3.21 Any preceding method, wherein step (2) comprises distillation, fractional distillation, chromatography, crystallization or a combination thereof.
3.22 Any preceding method, wherein the polymer is polystyrene, and wherein the method does not result in oxidation of either the methylene carbon atoms of the polystyrene backbone or any of the phenyl ring carbons of the polystyrene polymer.
3.23 Any preceding method, wherein the polymer is polystyrene, and wherein the product or products of the reaction comprises a compound of the formula:

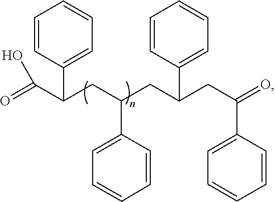

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
3.24 Any preceding method, wherein the polymer is polystyrene, and wherein 3.24 the product or products of the reaction comprises a compound of the formula:

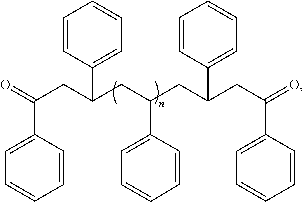

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof
3.25 Any preceding method, wherein the polymer is polystyrene, and wherein 3.25 the product or products of the reaction comprises a compound of the formula:

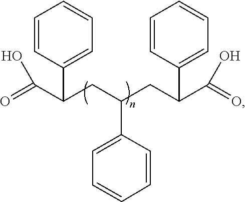

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
3.26 Method 3.23, wherein the method further comprises the step (3) of reducing the polystyrene oligomer's ketone groups to secondary alcohol groups.
3.27 Method 3.26, wherein the method further comprises the step (4) of reacting the resulting polystyrene oligomer acid-alcohols with a reagent to promote condensation and polymerization to form a homopolymeric polyester, and optionally isolating and/or purifying said polyester.
3.28 Method 3.26, wherein the method further comprises the step (4) of reacting the resulting polystyrene oligomer acid-alcohols with carboxylic acid/alcohol monomer (e.g., a terminal hydroxy alkane carboxylic acid) and a reagent to promote condensation and polymerization to form a heteropolymeric polyester, and optionally isolating and/or purifying said polyester.
3.29 Method 3.26, wherein the method further comprises the step (4) of reacting the resulting polystyrene oligomer acid-alcohols with carboxylic acid/amine monomer (e.g., a terminal amino alkane carboxylic acid) and a reagent to promote condensation and polymerization to form a heteropolymeric polyester-polyamide mixed copolymer, and optionally isolating and/or purifying said copolymer.
3.30 Method 3.24, wherein the method further comprises the step (3) of reducing the polystyrene oligomer's ketone groups to secondary alcohol groups.
3.31 Method 3.30, wherein the method further comprises the step (4) of reacting the resulting polystyrene oligomer dialcohol with dicarboxylic acid monomers (e.g., terminal carboxy alkane carboxylic acid) and a reagent to promote condensation and polymerization to form a heteropolymeric polyester, and optionally isolating and/or purifying said polyester.
3.32 Method 3.25, wherein the method further comprises the step (3) of reacting the resulting polystyrene oligomer diacid with a dialcohol monomer (e.g., a bis-terminal hydroxy alkane) and a regent to promote condensation and polymerization to form a heteropolymeric polyester, and optionally isolating and/or purifying said polyester;
3.33 Method 3.25, wherein the method further comprises the step (3) of reacting the resulting polystyrene oligomer diacid with a diamine monomer (e.g., a bis-terminal amino alkane) and a reagent to promote condensation and polymerization to form a heteropolymeric polyamide, and optionally isolating and/or purifying said polyamide.
3.34 Method 3.25, wherein the method further comprises the step (3) of reacting the resulting polystyrene oligomer diacid with an amino-alcohol monomer (e.g., a terminal amino alkane alcohol) and a reagent to promote condensation and polymerization to form a heteropolymeric mixed polyamide-polyester copolymer, and optionally isolating and/or purifying said copolymer.
3.35 Method 3.23, 3.24 or 3.25, further comprising any one or more steps necessary to form heteropolymeric polyimines or mixed polyester-polyimines, polyamide-polyimines, polyurethanes, or other heteropolymers.
3.36 Any preceding method, wherein the method further comprises one or more steps immediately following step (1) and before step (2), or after step (2) and before any subsequent steps, selected from peroxide passivation, further oxidation, downstream derivatization, catalytic scission, and catalytic rearrangement.
3.37 Any preceding method, wherein the ozone is generated using an ozone generator from an oxygen feed, and optionally wherein said oxygen is derived from the hydrolysis of water.

In a fourth aspect the present disclosure provides a method (Method 4) of making polymer-derived oligomers having terminal ketone and/or terminal carboxylic acid groups, the method comprising the steps of (1) exposing a polymer (e.g., a hydrocarbon polymer, such as polystyrene) to ozone, optionally in an aqueous and/or non-aqueous solvent, and (2) isolating and/or purifying the resulting product or products.

In further embodiments of the fourth aspect, the present disclosure provides as follows:
4.1 Method 4, wherein the polymer is a synthetic polymer, e.g., a hydrocarbon polymer, optionally a saturated hydrocarbon, a chlorinated hydrocarbon, or a polyamide (i.e., not a peptide polymer).
4.2 Method 4.1, wherein the polymer is a polyethylene, polypropylene, polystyrene, aliphatic polyamide (e.g., nylon, such as nylon-6,6), or a polyvinyl chloride or polyvinylidene chloride.
4.3 Method 4.2, wherein the polymer is polystyrene.
4.4 Method 4.3, wherein the polymer-derived oligomers have a structure selected from:

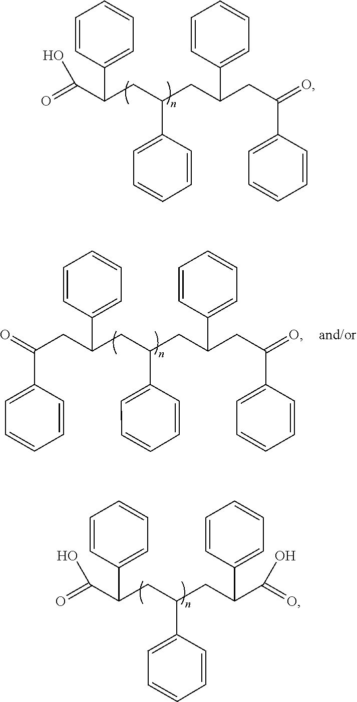

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
4.5 Method 4 or any of 4.1-4.4, wherein the step (1) of exposing the polymer (e.g., polystyrene) to ozone comprises exposing the polymer to an ozone/oxygen mixture or ozone/nitrogen mixture or ozone/air mixture.
4.6 Method 4 or any of 4.1-4.5, wherein step (1) is carried out in the absence of any other added oxidants or oxidizing agents.
4.7 Any preceding method, wherein step (1) does not comprise the presence or addition of any catalyst (e.g., any metal, activated charcoal, or silica gel).
4.8 Any preceding method, wherein step (1) occurs in the dark (e.g., the reaction occurs without exposure to light, e.g., UV light).
4.9 Any preceding method, wherein in step (1) the polymer (e.g., polystyrene) is dissolved or suspended in an aqueous solution or emulsion, optionally in an acidic (i.e., pH<7) or alkaline (e.g., pH>7) aqueous solution or emulsion.
4.10 Method 4.9, wherein the polymer (e.g., polystyrene) is dissolved or suspended in an alkaline aqueous solution, optionally wherein the alkaline agent is an inorganic base (e.g., an alkoxide, hydroxide, oxide, carbonate or bicarbonate of an alkali or alkaline earth metal).
4.11 Method 4.10, wherein the polymer (e.g., polystyrene) is dissolved or suspended in an aqueous solution of a sodium, potassium, lithium, calcium or magnesium hydroxide, alkoxide, oxide, carbonate or bicarbonate (e.g., sodium hydroxide or potassium hydroxide).
4.12 Method 4.10 or 4.11, wherein the aqueous solution or emulsion has a pH from 7.5 to 12, or from 8 to 12, or from 9 to 11, or from 9 to 10.
4.13 Any preceding method, wherein the polymer (e.g., polystyrene) is dissolved or suspended in a mixture of an aqueous solution and an organic co-solvent (such as an alcohol, ester, or ether solvent, e.g., methanol, ethanol, propanol, THF, or MTBE).
4.14 Any preceding method, wherein the products of the reaction (e.g., the polystyrene oligomers or carbonyl or carboxylic acid products) are obtained directly from the reaction between the polymer (e.g., polystyrene) and the ozone (e.g., no intermediate partially oxidized or oxidized species are formed or isolated).
4.15 Any preceding method, wherein the method does not comprise the formation of any alkyl peroxide intermediate.
4.16 Any preceding method, wherein the method does not comprise any step comprising a reducing agent between step (1) and step (2).
4.17 Method 4 or any of 4.1-4.16, wherein the method is a batch method.
4.18 Method 4 or any of 4.1-4.16, wherein the method is a continuous flow method, e.g., wherein the method is performed in a flow reactor.
4.19 Method 4 or any of 4.1-4.16, wherein the method is performed in one or more of a falling film reactor, a batch reactor, a continuous stirred-tank reactor, and/or loop reactor, either individually or in series.
4.20 Method 4.19, wherein the method is performed in one or more falling film reactors, e.g., multi-tube falling film reactors, optionally in series and optionally with recirculation, for example, as described in any embodiment of U.S. Pat. No. 10,071,944.
4.21 Any preceding method, wherein step (2) comprises separating the product or products from the reaction solvent, or from the ozone, or both.
4.22 Any preceding method, wherein step (2) comprises distillation, fractional distillation, chromatography, crystallization or a combination thereof.
4.23 Any preceding method, wherein the polymer is polystyrene, and wherein the method does not result in oxidation of either the methylene carbon atoms of the polystyrene backbone or any of the phenyl ring carbons of the polystyrene polymer.
4.24 Any preceding method, wherein the polymer is polystyrene, and wherein the product or products of the reaction comprises a compound of the formula:

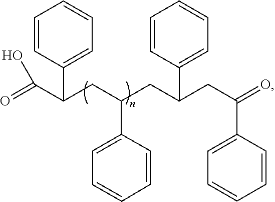

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
4.25 Any preceding method, wherein the polymer is polystyrene, and wherein 4.25 the product or products of the reaction comprises a compound of the formula:

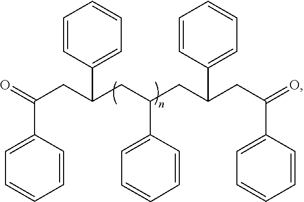

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof
4.26 Any preceding method, wherein the polymer is polystyrene, and wherein 4.26 the product or products of the reaction comprises a compound of the formula:

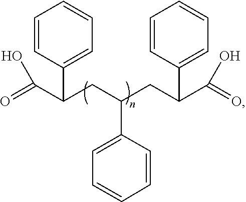

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.
4.27 Method 4.24, wherein the method further comprises the step (3) of reducing the polystyrene oligomer's ketone groups to secondary alcohol groups.
4.28 Method 4.27, wherein the method further comprises the step (4) of reacting the resulting polystyrene oligomer acid-alcohols with a reagent to promote condensation and polymerization to form a homopolymeric polyester, and optionally isolating and/or purifying said polyester.
4.29 Method 4.27, wherein the method further comprises the step (4) of reacting the resulting polystyrene oligomer acid-alcohols with carboxylic acid/alcohol monomer (e.g., a terminal hydroxy alkane carboxylic acid) and a reagent to promote condensation and polymerization to form a heteropolymeric polyester, and optionally isolating and/or purifying said polyester.
4.30 Method 4.27, wherein the method further comprises the step (4) of reacting the resulting polystyrene oligomer acid-alcohols with carboxylic acid/amine monomer (e.g., a terminal amino alkane carboxylic acid) and a reagent to promote condensation and polymerization to form a heteropolymeric polyester-polyamide mixed copolymer, and optionally isolating and/or purifying said copolymer.
4.31 Method 4.25, wherein the method further comprises the step (3) of reducing the polystyrene oligomer's ketone groups to secondary alcohol groups.
4.32 Method 4.31, wherein the method further comprises the step (4) of reacting the resulting polystyrene oligomer dialcohol with dicarboxylic acid monomers (e.g., terminal carboxy alkane carboxylic acid) and a reagent to promote condensation and polymerization to form a heteropolymeric polyester, and optionally isolating and/or purifying said polyester.
4.33 Method 4.26, wherein the method further comprises the step (3) of reacting the resulting polystyrene oligomer diacid with a dialcohol monomer (e.g., a bis-terminal hydroxy alkane) and a reagent to promote condensation and polymerization to form a heteropolymeric polyester, and optionally isolating and/or purifying said polyester;
4.34 Method 4.26, wherein the method further comprises the step (3) of reacting the resulting polystyrene oligomer diacid with a diamine monomer (e.g., a bis-terminal amino alkane) and a reagent to promote condensation and polymerization to form a heteropolymeric polyamide, and optionally isolating and/or purifying said polyamide.
4.35 Method 4.26, wherein the method further comprises the step (3) of reacting the resulting polystyrene oligomer diacid with an amino-alcohol monomer (e.g., a terminal amino alkane alcohol) and a reagent to promote condensation and polymerization to form a heteropolymeric mixed polyamide-polyester copolymer, and optionally isolating and/or purifying said copolymer.
4.36 Method 4.24, 4.25 or 4.26, further comprising any one or more steps necessary to form heteropolymeric polyimines or mixed polyester-polyimines, polyamide-polyimines, polyurethanes, or other heteropolymers.
4.37 Any preceding method, wherein the method further comprises one or more steps immediately following step (1) and before step (2), or after step (2) and before any subsequent steps, selected from peroxide passivation, further oxidation, downstream derivatization, catalytic scission, and catalytic rearrangement.
4.38 Any preceding method, wherein the ozone is generated using an ozone generator from an oxygen feed, and optionally wherein said oxygen is derived from the hydrolysis of water.

In a fifth aspect, the present disclosure provides compounds produced according to the methods disclosed herein, e.g., polymer-derived oligomers having terminal ketone and/or terminal carboxylic acid groups, for example, polystyrene-derived oligomers according to the following formulas:

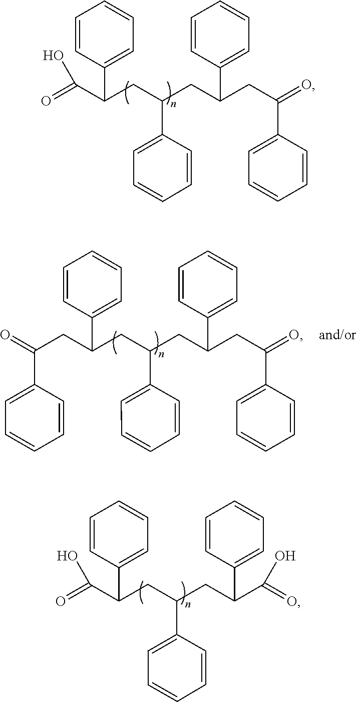

wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.

In another aspect, the present disclosure provides the compounds of the fourth and fifth aspects for use in a method of making polyester, polyamide, polyimide, polyepoxy, and polyurethane polymers, and mixed polymers comprising the aforementioned polymer types, as either homopolymers or heteropolymers.

The present disclosure further provides the use of these new polymers in the manufacture of dispersants, foam containers, packing peanuts, structural packing, biological containers for cell and tissue growth, food wrappers, 3-D printing, coatings, paints, lubricants, molded plastics, synthetic fibers, and personal care products such as hair products and cosmetics. The present disclosure also provides dispersants, foam containers, packing peanuts, structural packing, biological containers for cell and tissue growth, food wrappers, 3-D printing, coatings, paints, lubricants, molded plastics, synthetic fibers, and personal care products such as hair products and cosmetics comprising these new polymers.

While numerous process arrangements may be used to provide the polymer/ozone reactions (e.g., polystyrene/ozone reactions) described herein, the most preferred arrangement is a gas/liquid reaction in a highly controlled setting, such as that of a film on a temperature-controlled surface in a structured reactor. Therefore, in some embodiments, the methods described herein can be conducted in a Multi-Tube Film Reactor, of the type described in U.S. Pat. No. 10,071,944. The polymer (e.g., polystyrene) reactant can be dissolved in a suitable solvent or melted so as to have desirable flow properties for the reactor, and the polymer (e.g., polystyrene) solution or neat material can be circulated through the reactor once, or as many times as is required, and can also pass through one or more reactors in a sequence.

Following a suitable amount of time for reaction of the polymer (e.g., polystyrene) and ozone, additional steps may be provided, such as peroxide passivation, further oxidation, downstream derivatization, catalytic scission, and/or catalytic rearrangement.

As used herein, the word “polymer” is understood to carry its common meaning of a macromolecular large-molecular weight compound. In contrast, an “oligomer” refers to a much shorter, lower molecular weight compound, which can have as few as two repeating units (monomers). As used herein, these terms can overlap, because “oligomer” is used herein to refer to the deconstruction product according to the methods described herein from a polymer. Thus, a very small polymer (e.g., 5,000 monomer units) could be deconstructed into oligomers having 2 to 20 monomer units, while a very large polymer (e.g., 5,000,000 monomer units) could be deconstructed into oligomers having 2,000 to 20,000 monomeric units.

Nevertheless, for the sake of clarity, the term “polymers” as used herein means a polymer having at least 100 monomeric units. In some embodiments, the polymer will have at least 1,000 monomeric units, or at least 10,000 monomeric units, or at least 50,000 monomeric units, or at least 100,000 monomeric units, or at least 150,000 monomeric units, or at least 200,000 monomeric units, or at least 250,000 monomeric units, or at least 500,000 monomeric units, or at least 1,000,000 monomeric units.

As used herein, the term “oligomers” refers polymers and polymer derivatives having from 2 to 20,000 monomeric units, e.g., from 2 to 10,000, or from 2 to 5,000, or from 2 to 1,500, or from 2 to 1000, or from 2 to 500, or from 2 to 250, or from 2 to 100, or from 2 to 50, or from 2 to 25, monomeric units. In some embodiments, the oligomers described herein may have a number of monomeric units ranging from any one of 2, 3, 4, 5, 6, 7, 8, 9, 10, 12, 15, 20, 25, 30, 35, 40, 45, 50, 60, 70, 80, 90, 100, 125, 150, 175, 200, 225, 250, 300, 350, 400, 450, 500, 600, 700, 800, 900, 1000, 1250, 1500, 1750, 2000, 2500, 3000, 3500, 4000, 4500, 5000, 6000, 7000, 8000, 9000, 10,000, 15,000, 20,000, 25,000, 30,000, 40,000 or 50,000 units, up to any one or more of 10, 12, 15, 20, 25, 30, 35, 40, 45, 50, 60, 70, 80, 90, 100, 125, 150, 175, 200, 225, 250, 300, 350, 400, 450, 500, 600, 700, 800, 900, 1000, 1250, 1500, 1750, 2000, 2500, 3000, 3500, 4000, 4500, 5000, 6000, 7000, 8000, 9000, 10,000, 15,000, 20,000, 25,000, 30,000, 40,000 or 50,000, or 100,000 units.

In some embodiments, the polymer described herein may have an average molecular weight (e.g., a weight average or number average) of at least 5,000 Daltons, or at least 10,000 Daltons, or at least 50,000 Daltons, or at least 100,000 Daltons. In some embodiments, the polymers described herein have an average molecular weight (e.g., a weight average or number average) ranging from any one of 5,000 Daltons, 10,000 Daltons, 25,000 Daltons, 50,000 Daltons, 100,000 Daltons, 125,000 Daltons, 150,000 Daltons, 175,000 Daltons, 200,000 Daltons, 250,000 Daltons, 300,000 Daltons, 350,000 Daltons, 400,000 Daltons, 450,000 Daltons, 500,000 Daltons, 750,000 Daltons, 1M Daltons, 1.5M Daltons, 2M Daltons, 2.5M Daltons, 3M Daltons, 4M Daltons, or 5M Daltons, up to any one of 50,000 Daltons, 100,000 Daltons, 125,000 Daltons, 150,000 Daltons, 175,000 Daltons, 200,000 Daltons, 250,000 Daltons, 300,000 Daltons, 350,000 Daltons, 400,000 Daltons, 450,000 Daltons, 500,000 Daltons, 750,000 Daltons, 1M Daltons, 1.5M Daltons, 2M Daltons, 2.5M Daltons, 3M Daltons, 4M Daltons, 5M Daltons, 7.5M Daltons, 10M Daltons, 12.5M Daltons, 50M Daltons or 100M Daltons.

### Typical Conditions

Suitable solvents for step (1) of any of Method 1 et seq., Method 2 et seq., Method 3 et seq., Method 4 et seq., include apolar, polar protic and/or polar aprotic solvents, for example alcoholic solvents (e.g., methanol, ethanol, propanol, isopropanol, butanol). In some embodiments, the solvent for step (1) comprises an aqueous solution or emulsion, optionally an aqueous alkaline or aqueous acidic solution or emulsion. Any such aqueous solution may be a buffer. A buffer may be employed to maintain a pH>7. In some embodiments, the pH is between 7.5 and 12, or between 8 and 12, or between 9 and 11 or between 9 and 10. In some embodiments, the reaction occurs in an aqueous layer that forms an emulsion upon mixing with an organic layer. The organic layer may be the polymer (e.g., polystyrene) substrate (neat as a melted liquid) and/or a solution of the polymer (e.g., polystyrene) substrate in an organic solvent. In a preferred embodiment, an alkaline aqueous solution is combined with the neat polymer, such as, for example, a 2M NaOH solution mixed 1:1 (v/v or w/v) with neat polymer.

In some embodiments, the reaction is carried out at a temperature of −25° C. to 200° C. In a preferred embodiment, the reaction is run at 5° C. In some embodiments, the reaction is carried out for 0.1 to 100 hours. In a preferred embodiment the reaction is run for 2 hours.

In some embodiments, the ozonation is combined with electromagnetic irradiation to promote reactivity. In some embodiments, the wavelength is between 100-1000 nm, with a preferred embodiment between 200-280 nm. In other embodiments, the ozonation reaction is not exposed to any UV light, or is not exposed to any light (i.e., the reaction is in the dark).

1. A method of oxidizing the tertiary carbon (e.g., one or more tertiary carbons) of a polymer (e.g., a hydrocarbon polymer, such as polystyrene) using ozone, or of preparing at least one carbonyl compound (e.g., a ketone) or carboxylic acid from a polymer (e.g., a hydrocarbon polymer, such as polystyrene) using ozone, or of oxidatively decomposing a polymer to carbonyl and carboxylic acid degradation products, using ozone, or of making polymer-derived oligomers having terminal ketone and/or terminal carboxylic acid groups using ozone, the method comprising the steps of (1) exposing the polymer to ozone, optionally in an aqueous and/or non-aqueous solvent, and (2) isolating or purifying the resulting product or products.

2. A The method according to claim 1, wherein the method is a method of preparing at least one carbonyl compound (e.g., a ketone) or carboxylic acid from a polymer (e.g., a hydrocarbon polymer, such as polystyrene), comprising the steps of (1) exposing the polymer to ozone, optionally in an aqueous and/or non-aqueous solvent, and (2) isolating or purifying the resulting carbonyl and/or carboxylic acid product or products.

3. The method according to claim 1, wherein the method is a method of oxidatively decomposing a polymer (e.g., a hydrocarbon polymer, such as polystyrene) to carbonyl and carboxylic acid degradation products, comprising the steps of (1) exposing a polymer (e.g., a hydrocarbon polymer, such as polystyrene) to ozone, optionally in an aqueous and/or non-aqueous solvent, and optionally (2) isolating or purifying the resulting product or products.

4. The method according to claim 1, wherein the polymer is a synthetic polymer, e.g., a hydrocarbon polymer, optionally a saturated hydrocarbon, a chlorinated hydrocarbon, or a polyamide (i.e., not a peptide polymer).

5. The method according to claim 4, wherein the polymer is a polyethylene, polypropylene, polystyrene, aliphatic polyamide (e.g., nylon, such as nylon-6,6), or a polyvinyl chloride or polyvinylidene chloride.

6. The method according to claim 5, wherein the polymer is polystyrene.

7. The method according to claim 6, wherein the product or products of the reaction are selected from the following formulas: 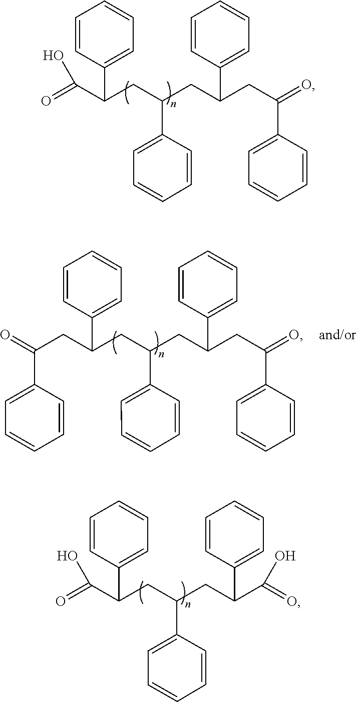
wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.

8. The method according to claim 1, wherein the method is a method of making polymer-derived oligomers having terminal ketone and/or terminal carboxylic acid groups, the method comprising the steps of (1) exposing a polymer (e.g., a hydrocarbon polymer, such as polystyrene) to ozone, optionally in an aqueous and/or non-aqueous solvent, and (2) isolating and/or purifying the resulting product or products.

9. The method according to claim 8, wherein the polymer is polystyrene, and the polystyrene-derived oligomers have a structure selected from: 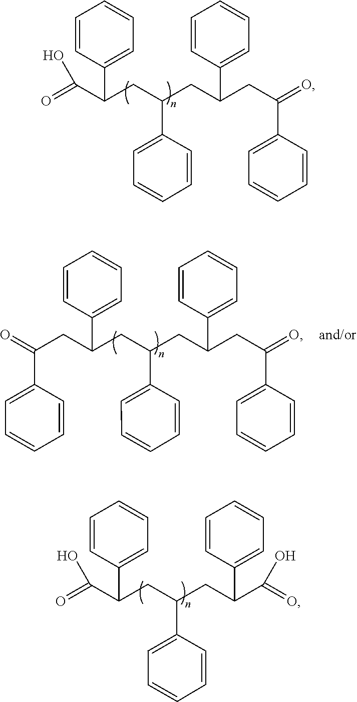
wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.

10. The method according to claim 1, wherein the step (1) of exposing the polymer (e.g., polystyrene) to ozone comprises exposing the polymer to an ozone/oxygen mixture or ozone/nitrogen mixture or ozone/air mixture.

11. The method according to claim 1, wherein step (1) is carried out in the absence of any other added oxidants or oxidizing agents.

12. The method according to claim 1, wherein step (1) does not comprise the presence or addition of any catalyst (e.g., any metal, activated charcoal, or silica gel).

13. The method according to claim 1, wherein step (1) occurs in the dark (e.g., the reaction occurs without exposure to light, e.g., UV light).

14. The method according to claim 1, wherein the method is a continuous flow method, e.g., wherein the method is performed in a flow reactor.

15. The method according to claim 14, wherein the method is performed in one or more of a falling film reactor, a batch reactor, a continuous stirred-tank reactor, and/or loop reactor, either individually or in series.

16. The method according to claim 14, wherein the method is performed in one or more falling film reactors, e.g., multi-tube falling film reactors, optionally in series and optionally with recirculation.

17. (canceled)

18. A compound according to one or more of the following formulas: 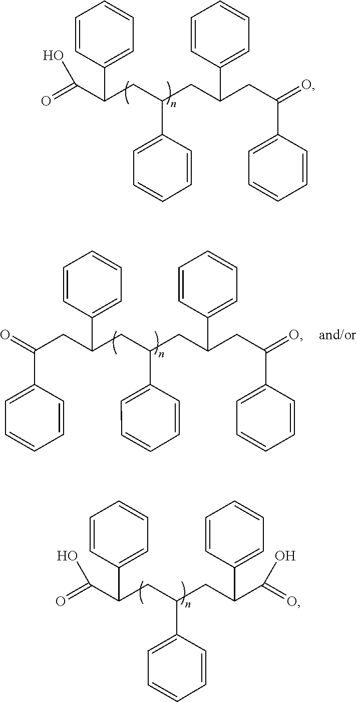
wherein n has a value from 0 to 100,000, e.g., 0 to 10,000, or 0 to 5,000, or 0 to 2,000, or 0 to 1,000, or 0 to 500, or 0 to 250, or 0 to 150, or 0 to 100, or 0 to 50, or 0 to 40, or 0 to 30, or 0 to 20, or 0 to 10, or 0 to 5, or any combination thereof.

19. The method according to claim 1, wherein the polymer is polystyrene, and wherein the method further comprises the step of reacting the product or products of the reaction with suitable reagents to form one or more of homopolymeric polyester polymers, heteropolymeric polyester polymers, heteropolymeric polyester-polyamide mixed copolymers, heteropolymeric polyamide polymers, heteropolymeric polyimine polymers or mixed polyester-polyimine copolymers, polyamide-polyimine copolymers, polyurethanes, or other heteropolymers.

20. A homopolymer or heteropolymer made according to the method of claim 19.

21. (canceled)

22. Dispersants, foam containers, packing peanuts, structural packing, biological containers for cell and tissue growth, food wrappers, 3-D printing, coatings, paints, lubricants, molded plastics, synthetic fibers, and personal care products such as hair products and cosmetics, comprising a homopolymer or heteropolymer made according to the method of claim 19.
